(19) United States
(12) Patent Application Publication
(10) Pub. No.: US 2007/0037861 A1
(43) Pub. Date: Feb. 15, 2007

(54) Enhanced method of treatment of growth disorders

(75) Inventors: Wayne Stephen Cutfield, Auckland (NZ); Paul Leslie Hofman, Auckland (NZ); Mark Hedley Vickers, One Tree Hill (NZ)

Correspondence Address: PHARMACIA CORPORATION;GLOBAL PATENT DEPARTMENT, POST OFFICE BOX 1027, ST. LOUIS, MO, 63006, US

(73) Assignee: PFIZER HEALTH AB, Stockholm (SE)

(21) Appl. No.: 10/572,699

(22) PCT Filed: Sep. 14, 2004
(86) PCT No.: PCT/IB04/03063

§ 371 Date: Jul. 10, 2006

(30) Foreign Application Priority Data
Sep. 19, 2003 (NZ) 528388

(51) Int. Cl. A61K 31/455; A61K 31/192

(52) U.S. Cl. 514/356; 514/571

(57) ABSTRACT

The application relates to the treatment of conditions and diseases for which growth hormone is a desirable method of treatment, using free fatty acid regulators in combination with growth hormone. In particular, the present invention discloses an enhanced method of treatment of growth disorders as well as methods to prevent and/or reduce adverse consequences of growth hormone treatment.

### FIELD OF INVENTION

The invention pertains to conditions and diseases for which growth hormone is a desirable method of treatment. In particular, the present invention discloses an enhanced method of treatment of growth disorders.

### BACKGROUND

Growth hormone (GH) therapy is used in the treatment of a variety of conditions. However, conventional GH therapy is subject to the presence of detrimental side effects. Side effects of GH therapy include glucose intolerance and/or diabetes, oedema, benign intracranial hypertension, arthralgia, myalgia, deterioration in glycaemic control in diabetic patients, paresthesias and carpal tunnel syndrome. Oedema is defined as an accumulation of an excessive amount of watery fluid in cells, tissues or serous cavities (such as the abdomen). Symptoms include puffiness of the face around the eyes, or in the feet, ankles and legs. GH induced salt and water retention can cause benign intracranial hypertension. Benign intracranial hypertension is characterized by increased cerebrospinal fluid pressure in the absence of a space occupying lesion. It can present with headache, visual loss, nausea, vomiting and papilloedema. Arthralgia is pain in one or more joints. Myalgia is pain or discomfort moving any muscle(s). Paresthesia is a term that refers to an abnormal burning or prickling sensation which is generally felt in the hands, arms, legs, or feet, but can occur in any part of the body. Carpal tunnel syndrome occurs when tendons or ligaments in the wrist become enlarged, often from inflammation. The narrowed tunnel of bones and ligaments in the wrist pinches the nerves that reach the fingers and the muscles at the base of the thumb. Symptoms range from a burning, tingling numbness in the fingers, especially the thumb and the index and middle fingers, to difficulty gripping or making a fist, to dropping things.

There has been some concern about the possibility of “cancer growth promotion” with growth hormone therapy, based upon a few cases of leukaemia reported in children treated with growth hormone therapy.

Growth hormone is known to antagonise the actions of insulin through multiple steps in the insulin-signalling cascade. GH therapy has been shown to impair insulin-mediated suppression of hepatic glucose output and increased peripheral glucose utilization (Sugimoto et al 1998). Some of the insulin antagonistic effects of GH are thought to be due to increased lipolysis and subsequent elevation in plasma free fatty acids (FFA) leading to inhibition of glucose uptake (Moller et al 1987). An increase in circulating FFA is associated with a reduction in insulin sensitivity as FFAs are known to impair insulin mediated glucose uptake in skeletal muscle (Felber et al 1964, Reaven et al 1988, Randle et al 1963).

The diabetogenic effects of GH therapy during childhood have recently been highlighted. An increased incidence of type 2 diabetes mellitus in children and adolescents during GH therapy has been found in populations at greatest risk of the disease (Cutfield et al 2000). Adult males born of low birth weight have an increased incidence of type 2 diabetes mellitus, dyslipidemia and hypertension (Barker et al 1993, Barker 1994, Law et al 1991).

It has been shown that prepubertal short children exhibiting intrauterine growth retardation (IUGR) have markedly reduced insulin sensitivity, i.e. they are insulin resistant, compared to short children of normal birth weight (Hofman et al 1997). Girls with Turner syndrome have also been shown to exhibit reduced insulin sensitivity when compared to normal girls (Caprio et al 1991).

Reduced insulin sensitivity or secondary hyperinsulinism has been implicated in the pathogenesis of all the above mentioned disorders (Reaven et al 1991). Insulin resistance has been found to be a marker of type 2 diabetes mellitus in those at risk of type 2 diabetes (Martin et al 1992). In non-diabetic, euglycemic humans and animals fasting hyperinsulinemia reflects a generalised increase in insulin secretion that is a compensatory response for a reduction in insulin sensitivity Kahn et al 1993). In addition, insulin resistance is involved in the pathogenesis of hypertension.

Insulin resistance and secondary hyperinsulinism are important in the pathogenesis of hypertension which occurs more commonly in adults of low birth weight (Barker et al 1993, Law et al 1991). Insulin has an important vasodilatory function that is mediated through nitric oxide release (McNally et al 1995, Steinberg et al 1994). Insulin-induced vasodilation is impaired in disorders characterised by insulin resistance (Laakso et al 1992, Laakso et al 1993, Feldman et al 1993).).

The applicants have previously observed that in IUGR children the marked reduction in insulin sensitivity that occurred during GH therapy was still present 3 months after stopping treatment (Cutfield, et al 2000 (2)).

In the light of the above observations it is clearly advantageous to establish a method of eliminating or at least alleviating the side effects of the GH treatment of growth disorders. It would be particularly advantageous to establish a method combining GH replacement therapy with a compound that produces synergy in the somatogenic effects of standard GH therapy, but reduces its undesirable side-effects.

### SUMMARY OF THE INVENTION

This invention is directed at the use of combination therapy comprising growth hormone (GH) and at least one free fatty acid (FFA) regulator in the treatment of conditions that require or have the potential to require treatment with GH.

In particular, the invention is directed at methods of GH treatment, whereby the somatogenic effects of GH treatment are enhanced and some of the metabolic and lactogenic side effects of hGH treatment are reduced.

More particularly, the invention is directed at treatment ofjuvenile patients in the need to growth hormone replacement therapy.

In one embodiment, the invention provides a method for treating a growth disorder in a mammal, said method comprising administering to said mammal an effective amount of at least one FFA regulator in combination with growth hormone. In a preferred embodiment, said mammal is a human. In another preferred embodiment, said mammal is a juvenile, more preferably a child or adolescent.

In another embodiment, the invention provides a method of increasing the growth promoting effects of growth hormone therapy in a mammal, said method comprising administering to said mammal an effective amount of at least one FFA regulator in combination with growth hormone. In a preferred embodiment, said mammal is a human. In another preferred embodiment, said mammal is a juvenile, more preferably a child or adolescent.

In still another embodiment, the invention provides a method of preventing or treating an adverse consequence of growth hormone treatment, preferably of a growth disorder, in a mammal, comprising administering an effective amount of at least one FFA regulator in combination with growth hormone. In a preferred embodiment, said adverse consequence of GH treatment is oedema. In another preferred embodiment, said adverse consequence of GH treatment is trabecular bone loss. In still another preferred embodiment, said mammal is a human. In yet another preferred embodiment, said mammal is a juvenile, more preferably a child or adolescent.

In still another embodiment, the invention relates to the use of a combination of growth hormone and at least one FFA regulator in the preparation of a medicament or composition for treating growth disorders in a mammal. In a preferred embodiment, said mammal is a human. In another preferred embodiment, said mammal is a juvenile, more preferably a child or adolescent.

In still another embodiment, the invention relates to the use of at least one FFA regulator in the preparation of a medicament for increasing the growth promoting effects of growth hormone therapy in a mammal. In a preferred embodiment, said mammal is a human. In another preferred embodiment, said mammal is a juvenile, more preferably a child or adolescent. In still another embodiment, said medicament comprises a combination of said growth hormone and said FFA regulator(s).

In still another embodiment, the invention relates to the use of at least one FFA regulator in the preparation of a medicament for preventing or treating an adverse consequence of growth hormone treatment in a mammal, preferably in a mammal suffering from a growth disorder. In a preferred embodiment, said adverse consequence of GH treatment is oedema. In another preferred embodiment, said adverse consequence of GH treatment is trabecular bone loss. In still another preferred embodiment, said mammal is a human. In yet another preferred embodiment, said mammal is a juvenile, more preferably a child or adolescent. In still another embodiment, said medicament comprises a combination of said growth hormone and said FFA regulator(s).

In yet further embodiments, this invention includes compositions suitable for the practice of the methods and uses of the invention. In particular, the invention provides a composition or medicament for treating growth disorders and/or preventing or treating the adverse consequences of growth hormone treatment, said composition or medicament comprising growth hormone and at least one FFA regulator. In one embodiment of the invention, said FFA regulator is fibric acid or a fibric acid derivative, preferably fenofibrate. In another embodiment of the invention, said FFA regulator is nicotinic acid or a nicotinic acid derivative, preferably acipimox.

In any of the method, use or composition of the invention, administration of said FFA regulator(s) may occur prior to, in combination with or following growth hormone administration.

### DETAILED DESCRIPTION OF FIGURES

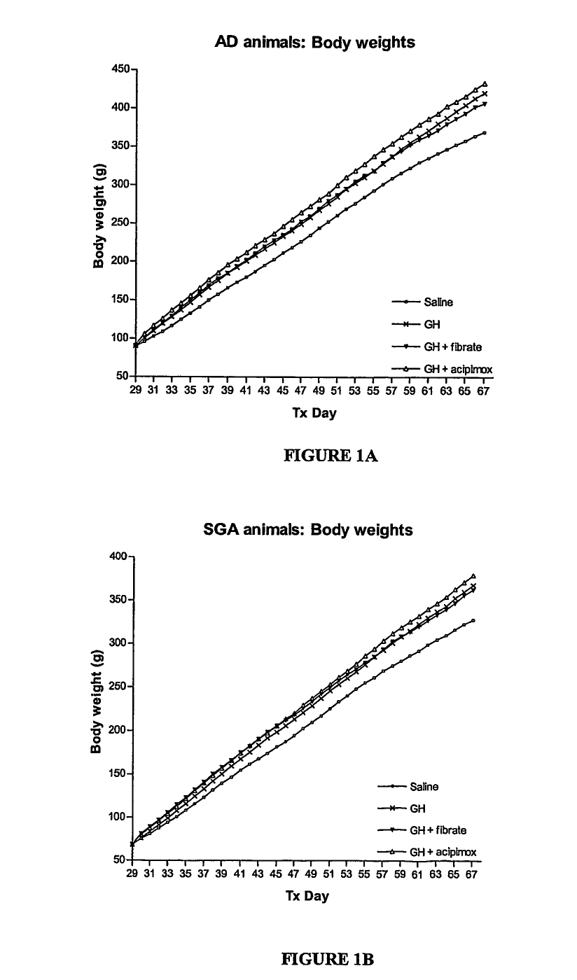

FIG. 1 depicts the body weight gain curves for each treatment sub-groups: in the ad libilum (AD) group (FIG. 1a) and the small for gestational age group (SGA) (FIG. 1b).

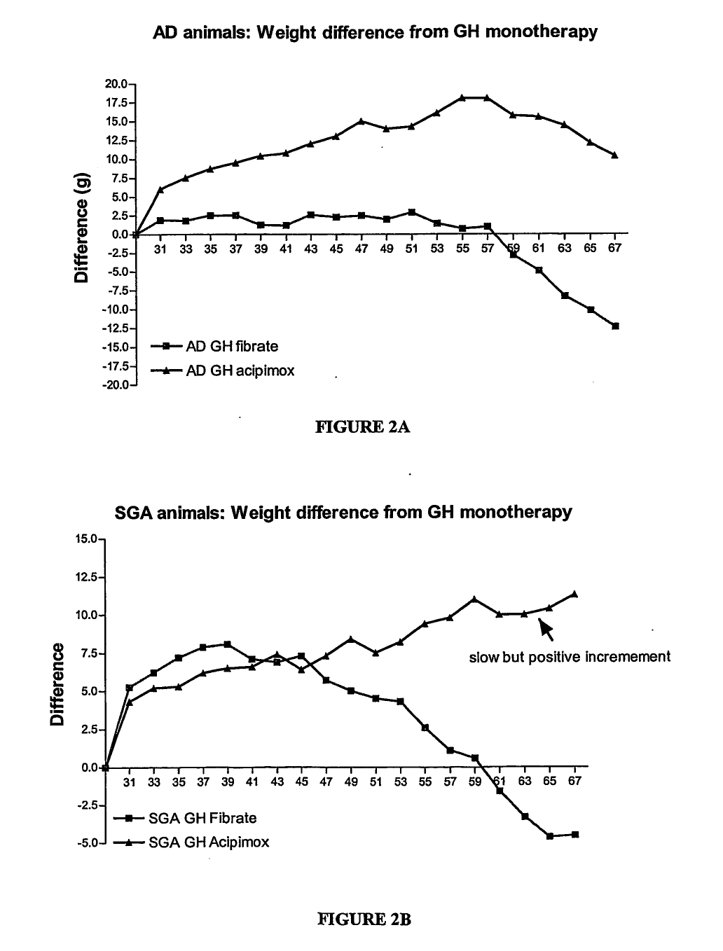

FIG. 2 depicts the weight gain differential from animals treated with GH alone: for AD animals (FIG. 2a) and for SGA animals (FIG. 2b).

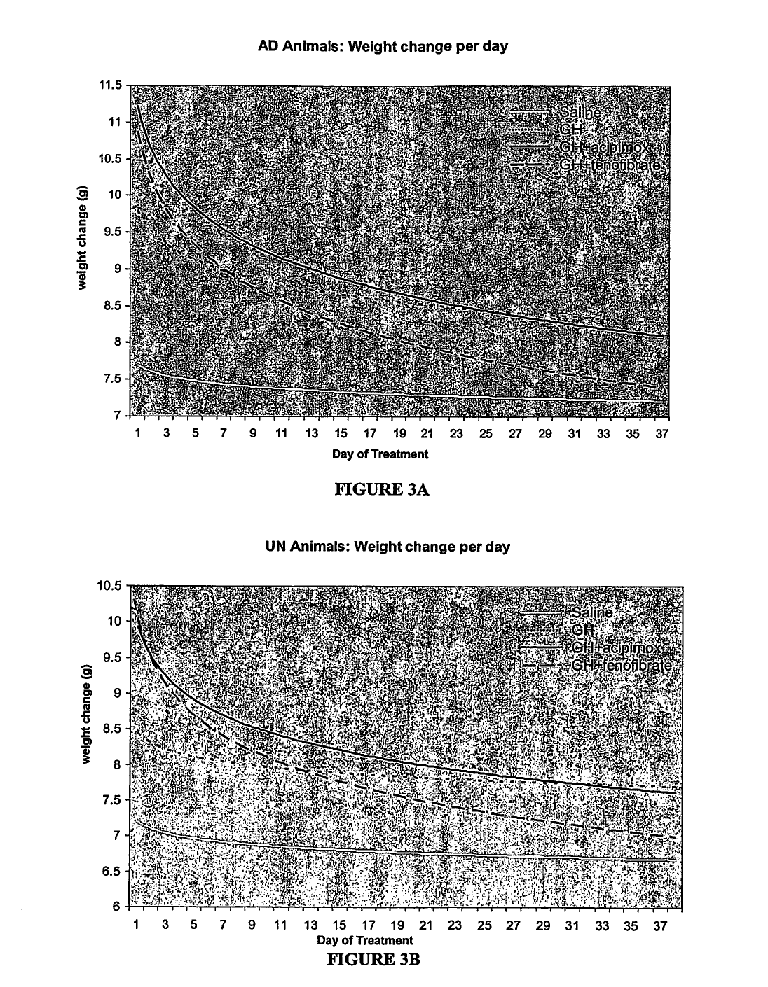

FIG. 3 depicts the daily changes in body weight (AD animals in FIG. 3a; SGA animals in FIG. 3b). Bottom axis is day of treatment.

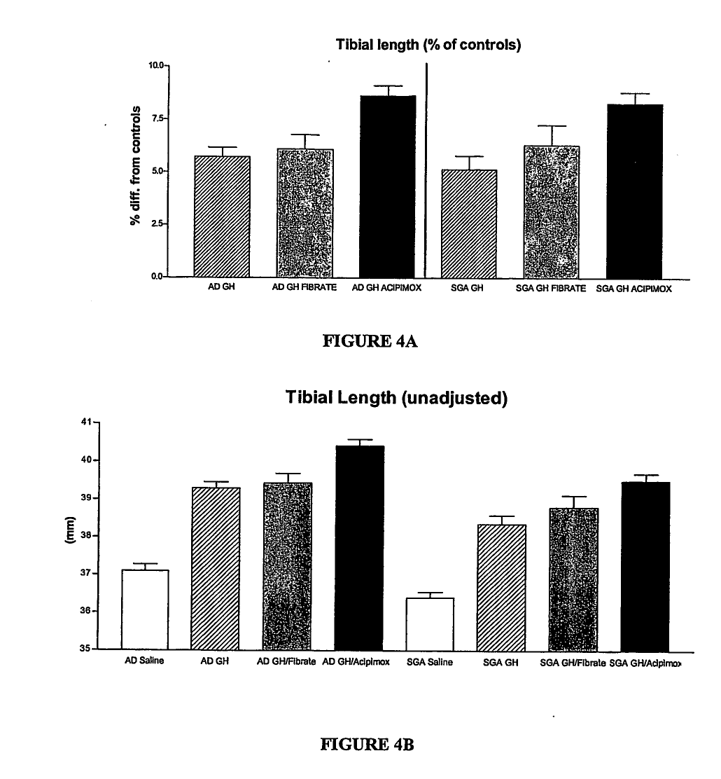

FIG. 4a depicts tibial length change as a percentage of change in the saline treated group for both AD and SGA animals. FIG. 4b represents unadjusted tibial length across all treatment groups.

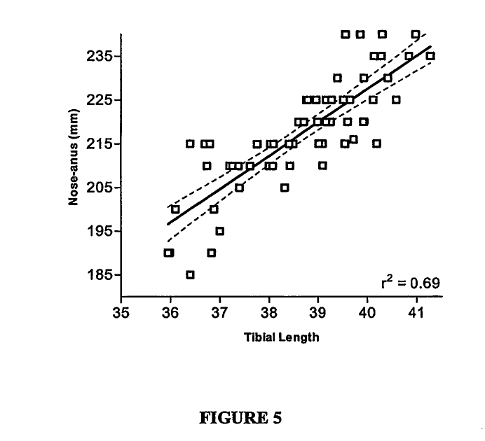

FIG. 5 depicts the relationship between total body length (nose-anus) and tibial bone length.

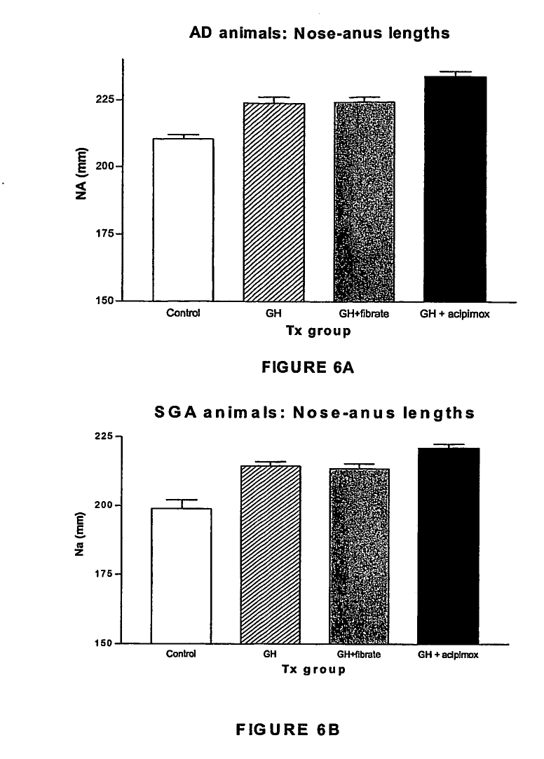

FIGS. 6a and 6b show anus to nose lengths of the AD (FIG. 6a) and SGA (FIG. 6b) groups post-mortem.

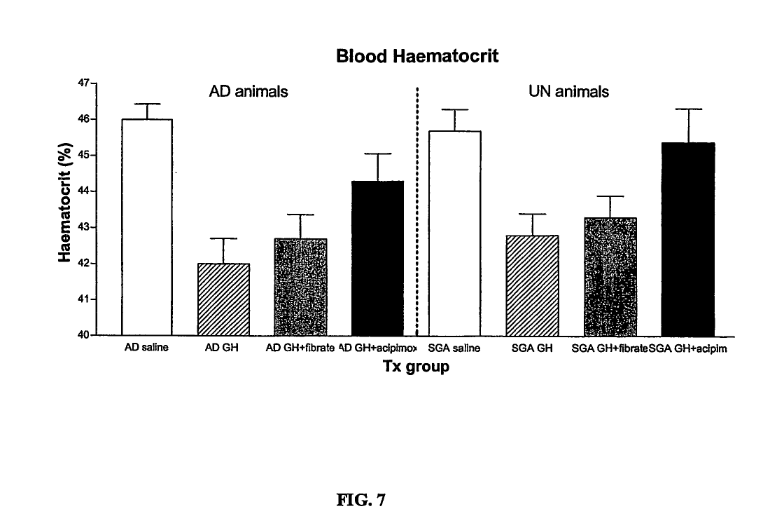

FIG. 7 depicts the effects of the each treatment in AD and undernourished (UN) groups on blood haematocrit.

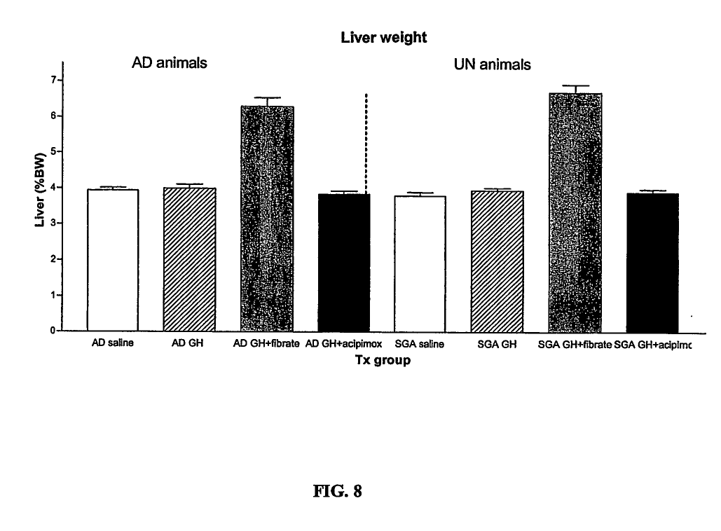

FIG. 8 depicts changes in liver weights in each treatment groups as a percentage of a total body weight.

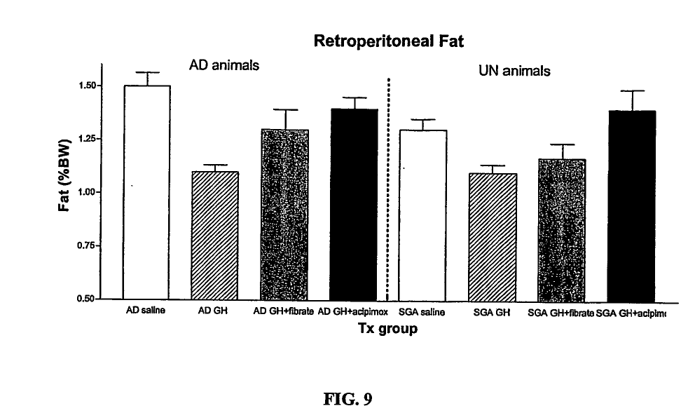

FIG. 9 depicts retroperitoneal fat mass in each treatment group as a percentage of total body weight.

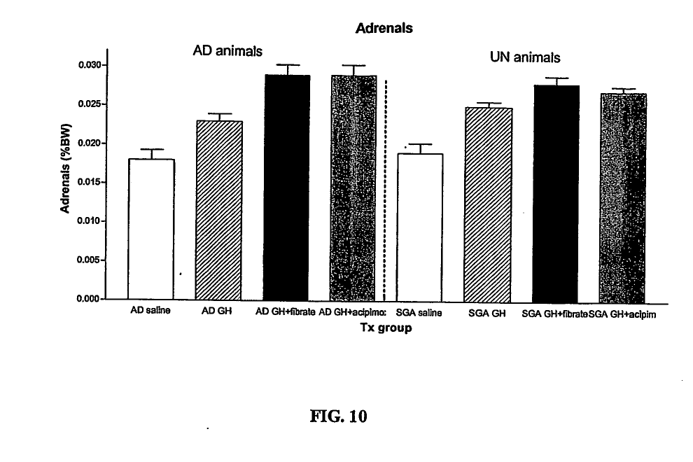

FIG. 10 depicts adrenal weights in each treatment group as a percentage of total body weight.

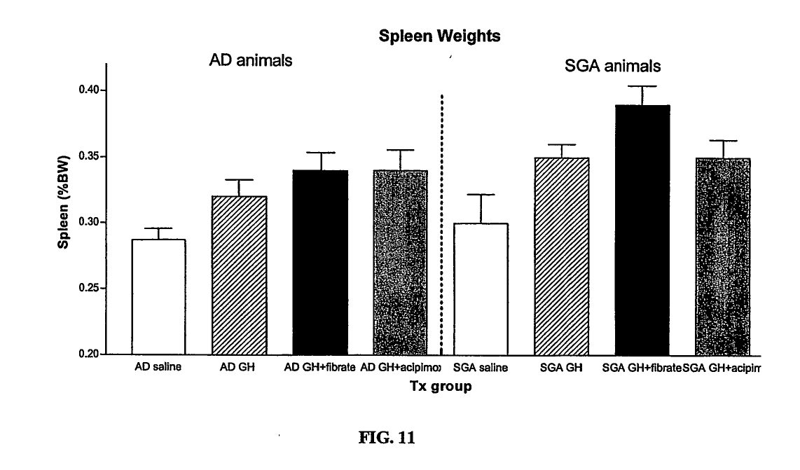

FIG. 11 depicts spleen weights in each treatment group as a percentage of total body weight.

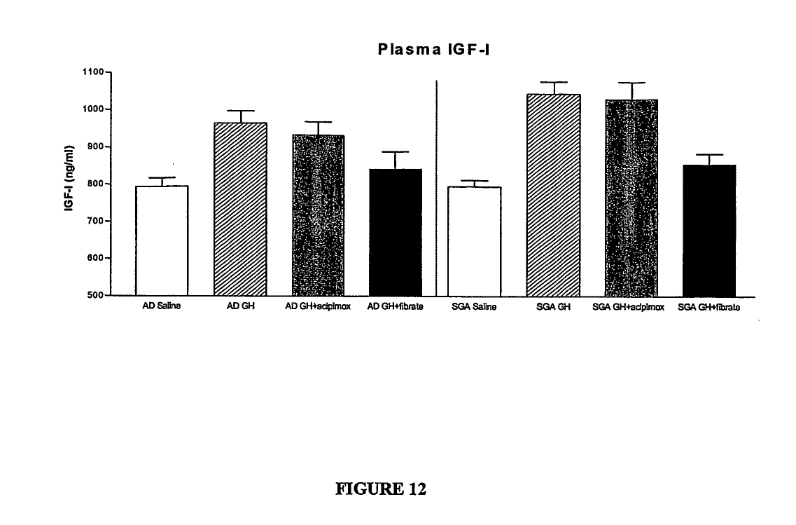

FIG. 12 depicts plasma IGF-I concentrations in each treatment group at time of sacrifice.

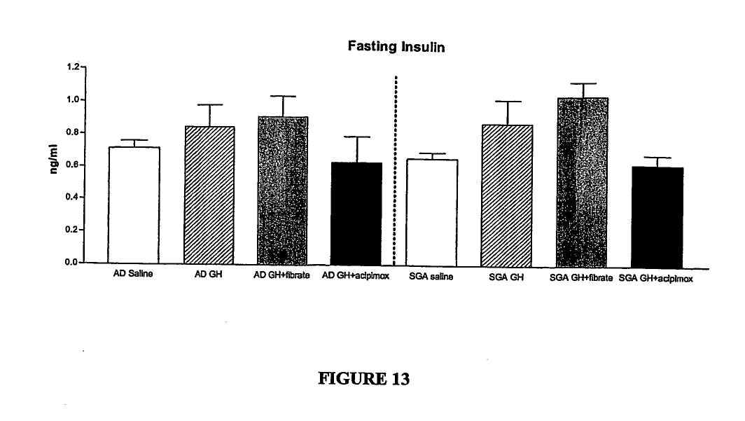

FIG. 13 depicts plasma insulin concentrations in each treatment group following an overnight fast.

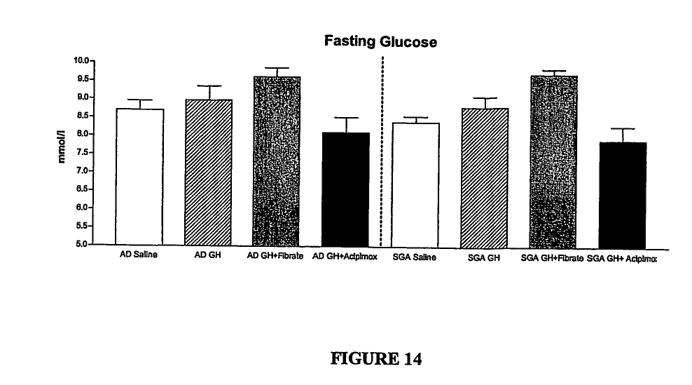

FIG. 14 depicts fasting plasma glucose concentrations in each treatment group.

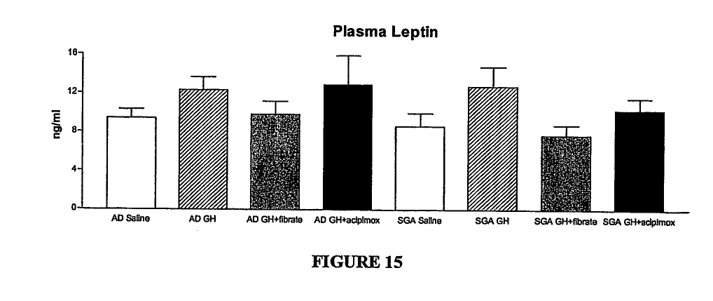

FIG. 15 depicts plasma leptin concentrations in each treatment group at completion of trial

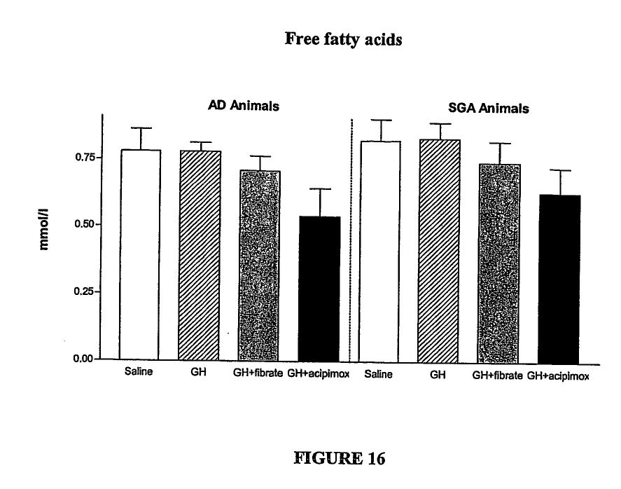

FIG. 16 depicts plasma free fatty acids (FFAs) levels in each treatment group following an overnight fast.

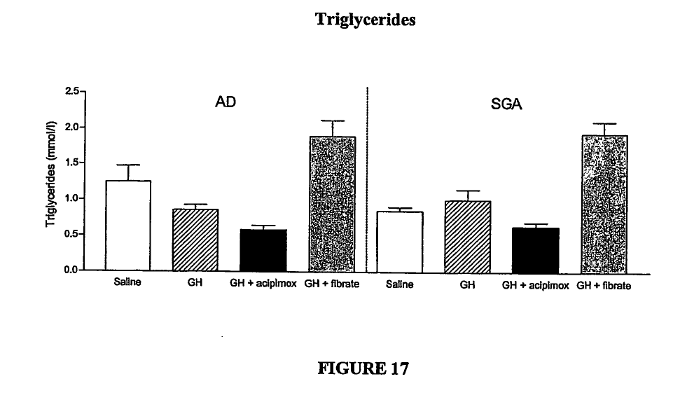

FIG. 17 depicts plasma triglycerides in each treatment group following an overnight fast.

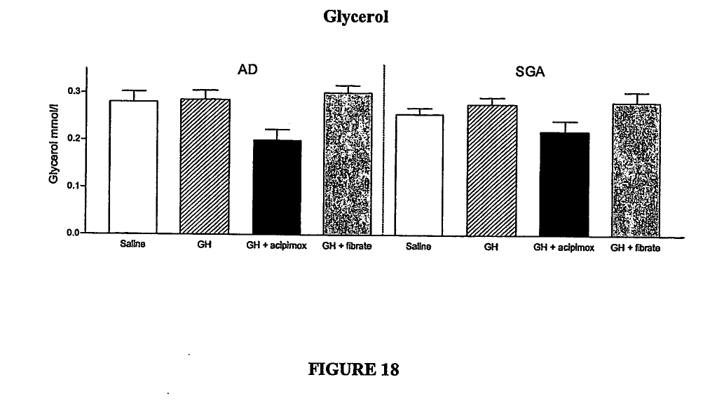

FIG. 18 depicts plasma free glycerol in each treatment group.

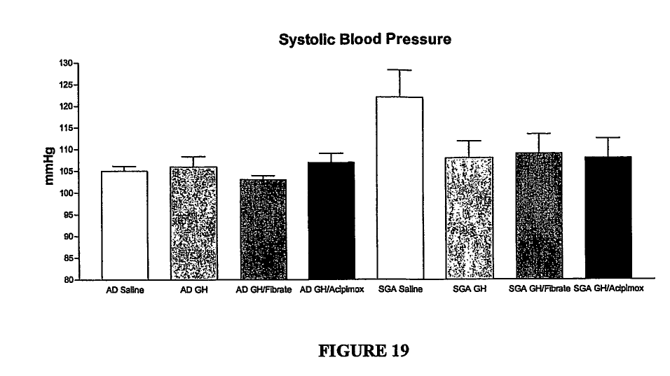

FIG. 19 depicts systolic blood pressure in each treatment group.

### DETAILED DESCRIPTION OF THE INVENTION

Definitions

As used herein, the term ‘growth hormone’ or ‘GH’, includes growth hormone; growth hormone secretagogues (GHSs); growth hormone releasing proteins/peptides (GHRP); growth hormone releasing hormone (GHRH); somatotropin release inhibitory factor (SRIF); compounds which increase the endogenous release of growth hormone or growth hormone secretagogues; a pharmaceutically acceptable salt of a GHS; analogues; mimetics; functionally equivalent ligands; prodrugs; metabolites; derivatives; agonists; compounds which increase the activity of neural growth hormone receptors; compounds which bind to or increase the concentration of compounds which bind to neural growth hormone receptors; compounds which lessen or prevent inhibition of GH, GHS or ligand activity; or inhibitors of antagonists thereof.

Examples of agents which stimulate growth hormone and production or lessen or prevent its inhibition include, but are not limited to, growth hormone releasing peptides such as GHRP-1, GHRP-2 (also known as KP-102), GHRP-6, hexarelin, G-7039, G-7502, L-692,429, L-629,585, L-163,191 (aka MK-0677), ipamorelin, NN₇O₃, GHS-25, CP-424,391, ghrelin, SM-130686 or GHRH or inhibitors of GH antagonists (substances which bind growth hormone or otherwise prevent or reduce the action of GH within the body). These latter compounds exert an indirect effect on effective GH concentrations through the removal of an inhibitory mechanism and include substances such as somatostatin release inhibitory factor (SRIF).

The GH can be any GH in native-sequence or in variant form and from any source, whether natural, synthetic or recombinant. Examples being human GH, bovine GH, rat GH and porcine GH. It is, however, preferred that the GH employed be human GH and more preferably recombinant human GH. Examples of human growth hormone include but are not limited to human growth hormone (hGH), which is natural or recombinant GH with the human native sequence (for example, GENOTROPIN™, somatotropin or somatropin), and recombinant growth hormone (rGH), which refers to any GH or GH variant produced by means of recombinant DNA technology, including recombinant human native-sequence, mature GH with or without a methionine at its N-terminus, somatrem, somatotropin, and somatropin. Another example is methionyl human growth hormone (met-hGH) produced in E. coli, e.g., by the process described in U.S. Pat. No. 4,755,465 issued Jul. 5, 1988 and Goeddel et al., Nature, 282: 544 (1979). Met-hGH, sold as PROTROPIN™ (Genentech, Inc. U.S.A.), which is identical to the natural polypeptide, with the exception of the presence of an N-terminal methionine residue. Another example is recombinant hGH sold as NUTROPIN™ (Genentech, Inc., U.S.A.). This latter hGH lacks this methionine residue and has an amino acid sequence identical to that of the natural hormone. See Gray et al., Biotechnology 2: 161 (1984). Another GH example is an hGH variant that is a placental form of GH with pure somatogenic and no lactogenic activity as described in U.S. Pat. No. 4,670,393. Also included are GH variants, for example such as those described in WO 90/04788 and WO 92/09690.

In a particular embodiment, the GH molecule or GH variant thereof is modified, preferably is pegylated.

As used herein “treatment” of a disease or “therapy” for it includes preventing the disease from occurring in a mammal that may be predisposed to the disease but does not yet experience or exhibit symptoms of the disease (prophylactic treatment), inhibiting the disease (slowing or arresting its development), providing relief from the symptoms or side effects of the disease, and relieving the disease (causing regression of the disease).

As used herein, the term “adverse consequence of growth hormone treatment” refers to any side effects or adverse events resulting from a growth hormone treatment. This term therefore includes but is not limited to the following: glucose intolerance, insulin resistance, secondary hyperinsulinism, diabetes, dyslipidemia, hypertension, obesity, conditions associated with sodium and water retention including oedema; trabecular bone loss, benign intracranial hypertension, arthralgia, myalgia, deterioration in glycaemic control in diabetic patients, paresthesias and carpal tunnel syndrome. Preferably, the invention relates to the treatment of oedema and/or trabecular bone loss.

As used herein, the term “free fatty acid (FFA) regulator” refers to any compound that has a hypolipidemic effect i.e. lowers FFA levels. The FFA regulators of interest include but are not limited to fibric acid and derivatives thereof, and nicotinic acid (niacin) and derivatives thereof. The effects of fibrates are mediated by activation of peroxisome proliferators-activated receptors (PPAR). PPARα is thought to mediate the hypotriglyceridemic effect of fibrates by stimulating catabolic pathways of fatty acids in the liver. PPARα activators also decrease adipose tissue mass. Fenofibrate, ciprofibrate and GW9578 have been found to reduce insulin resistance without adverse effects on body weight and adipose tissue mass in an animal model. PPARα agonists may exert direct insulin-sensitising actions. Bezafibrate has been shown to reduce fat deposits and improve insulin sensitivity. In adipocytes, nicotinic acid reduces lipolysis by inhibiting adenylyl cyclase, resulting in the suppression of hormone-sensitive lipase (Holm et al., (2000) Molecular mechanisms regulating hormone-sensitive lipase and lipolysis. Annu Rev Nutr 20:365-393). Overnight administration of acipimox, a long-acting analog of nicotinic acid, was shown to inhibit lipolysis and lower plasma FFA levels, reduce insulin resistance, increase carbohydrate oxidation, improve oral glucose tolerance, and reduce plasma insulin levels in lean and obese nondiabetic subjects and subjects with impaired glucose tolerance or type 2 diabetes (Santomauro et al. (1999) Overnight lowering of free fatty acids with acipimox improves insulin resistance and glucose tolerance in obese diabetic and nondiabetic subjects. Diabetes 48:1836-1841). The fibric acid derivatives include, but are not limited to, fenofibrate, clofibrate, gemfibrozil, bezafibrate and ciprofibrate. The nicotinic acid (niacin) derivatives include but are not limited to extended-release niacin; controlled-release niacin; niacinamide (nicotinamide); acipimox (5-methylpyrazinecarboxylic acid 4-oxide); and nicotinic acid esters (methyl nicotinate, hexyl nicotinate), niceritrol, acifran, cyclohexylphenyl nicotinate, and cyclohexylphenyl-oxide nicotinate.

As used herein, the terms “co-administration”, “co-administered” and “in combination with”, referring to growth hormone and one or more free fatty acid regualorts, is intended to mean, and does refer to and include the following:
simultaneous administration of such combination of GH and FFA regulator(s) to a patient in need of treatment, when such components are formulated together into a single dosage form which releases said components at substantially the same time to said patient,
substantially simultaneous administration of such combination of GH and FFA regulator(s) to a patient in need of treatment, when such components are formulated apart from each other into separate dosage forms which are taken at substantially the same time by said patient, whereupon said components are released at substantially the same time to said patient
sequential administration of such combination of GH and FFA regulator(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of GH and FFA regulator(s) to a patient in need of treatment, when such components are formulated together into a single dosage form which releases said components in a controlled manner whereupon they are concurrently, consecutively, and/or overlappingly administered at the same and/or different times by said patient.
‘Somatogenic effects’ of hGH treatment include, but are not limited to the growth-promoting, body-weight increasing and osteo-anabolic actions.
‘Lactogenic effects’ of hGH treatment include, but are not limited to the effects of exogenous growth hormone that are associated with prolactin receptor (PRLR) signalling. Those effects include but not limited to: mammary gland development, changes in osmotic balance and cell proliferation.
‘Metabolic effects’ of hGH treatment include, but are not limited to stimulation of lipolysis, stimulation of secretion of IGF-1, and diabetogenic effects.

Conditions Treated Using GH

Conditions treated using GH include growth disorders as well as adult growth hormone deficiency (aGHD), chronic renal insufficiency (CRI), Aids wasting, Aging, Erectile dysfunction, HIV lipodystrophy, Fibromyalgia, Osteoporosis, Memory disorders, Depression, Crohn's disease, Traumatic brain injury, Subarachnoid haemorrhage, Noonan's syndrome, End stage renal disease (ESRD), Bone marrow stem cell rescue, Metabolic syndrome, and Glucocorticoid myopathy.

As used herein, the term “growth disorder” refers to any condition resulting in short stature. Such conditions include but are not limited to growth hormone insufficiency, growth hormone deficiency (GHD), intrauterine growth retardation (IUGR), growth failure in children who were born small for gestional age (SGA), very low birth weight (VLBW), skeletal abnormalities including dysplasias, chromosomal variations (Turner's Syndrome, Down Syndrome, Prader-Willi Syndrome), chronic renal insufficiency related growth retardation, constitutional delay of growth, cystic fibrosis related growth retardation, idiopathic short stature (ISS), short stature due to glucocorticoid treatment in children, failure of growth catching for short premature children, or any other condition resulting in short stature.

GH Deficiency

Diagnosis of growth hormone deficiency requires growth hormone stimulation testing. Tests used include the insulin hypoglycemia test or insulin tolerance test (ITT), L-dopa stimulation test, arginine infusion test and arginine/GHRH test. Peak growth hormone secretion levels in adults of less than 3-5 ng/mL are indicative of GHD. In children values below 10 ng/mL are considered inadequate. Growth hormone deficiency is treated with recombinant human growth hormone which is usually given via a subcutaneous injection on a daily basis.

There are several causes of GHD in children and most can be related to a problem in the hypothalamus or the pituitary. In certain rare cases, a defect in the body's utilization of growth hormone occurs. In most children with growth hormone deficiency, the defect lies in the hypothalamus. When other pituitary hormones are also not being secreted normally, the child is said to have hypopituitarism. In congenital hypopituitarism, abnormal formation of the pituitary or hypothalamus occurs during fetal development. Acquired hypopituitarism results from damage to the pituitary or hypothalamus that occurs during or following birth. It can be caused by a severe head injury, brain damage due to disease, radiation therapy, or a tumour.

The worldwide incidence of GHD in children has been estimated to be at least 1 in 10,000 live births and some individual countries have reported an incidence as high as 1 in 4,000 live births. A growth hormone deficient child usually shows a growth pattern of less than 2 inches a year. In many cases the child will grow normally until the age of 2 or 3 and then begin to show signs of delayed growth. Testing for growth hormone deficiency will occur when other possibilities of short stature have been ruled out. A weekly dose of up to 0.30 mg/kg of body weight divided into daily subcutaneous injections is recommended for GHD children.

In adults, deficiency of growth hormone can develop in the following situations; presence of a large pituitary tumour, after surgery or radiation therapy of pituitary tumour or other brain tumours, secondary to hypothalamic disorders and the continuation of childhood growth hormone deficiency into adulthood. The clinical features of adult GHD include; fatigue, muscle weakness, reduced exercise capacity, weight gain, increase in body fat and decrease in muscle mass, increase in LDL cholesterol and triglycerides and decrease in HDL cholesterol, increased risk for heart attack, heart failure and stroke, decrease in bone mass, anxiety and depression, especially lack of sense of well-being, social isolation and reduced energy. In the United States, an estimated total of 35,000 adults have GHD and approximately 6,000 new cases of GHD occur each year. For the average 70 kg man, the recommended dosage at the start of therapy is approximately 0.3 mg given as a daily subcutaneous injection. The dose can be increased, on the basis of individual requirements, to a maximum of 1.75 mg daily in patients younger than 35 years of age and to a maximum of 0.875 mg daily in patients older than 35 years. Lower doses may be needed to minimize the occurrence of adverse events, especially in older or overweight patients.

Prader-Willi Syndrome

Prader-Willi syndrome is a disorder of chromosome 15 characterised by hypotonia, hypogonadism, hyperphagia, cognitive impairment and difficult behaviour; the major medical concern being morbid obesity. Growth hormone is typically deficient, causing short stature, lack of pubertal growth spurt, and a high body fat ratio, even in those with normal weight. The need for GH therapy should be assessed in both children and adults. In children, if growth rate falls or height is below the third percentile, GH treatment should be considered. Growth hormone replacement helps to normalize the height and increases lean body mass; these both help with weight management. The usual weekly dose is 0.24 mg/kg of body weight; this is divided into 6 or 7 smaller doses over the course of the week.

Turner Syndrome

Turner syndrome occurs in approximately 1 in 2,500 live-born girls. It is due to abnormalities or absence of an X chromosome and is frequently associated with short stature, which can be ameliorated by GH treatment. Other features of Turner syndrome can include shortness of the neck and at times, webbing of the neck, cubitus valgus, shortness of fourth and fifth metacarpals and metatarsals, a shield shaped chest and primary hypogonadism. Growth in height is variable in patients with Turner syndrome so the decision whether to treat with GH and the timing of such treatment is made on an individual basis. Often, treatment is initiated when a patient's height declines below the 5^{th} percentile or when the standard deviation score decreases to less than 2 standard deviations below the mean. Treatment is often initiated with GH doses slightly higher than those used in treating GHD; a common starting dosage is 0.375 mg/kg per week divided into daily doses.

Chronic Renal Insufficiency

Chronic renal insufficiency (CRI) affects about 3,000 children in the United States. It manifests through a gradual and progressive loss of the ability of the kidneys to excrete wastes, concentrate urine, and conserve electrolytes. Approximately a third of children with chronic renal disease have abnormal growth partly because renal diseases disturb the metabolism of growth hormone. The corticosteroid hormones which are often used to treat the kidney disease can also retard growth. Kidney transplants can help a child start growing normally again, but most children do not make up the growth lost prior to transplantation. The age that the renal disease starts has more impact on growth retardation than the reduction in renal function (i.e. the younger the child when the disease starts, the more retarded is his or her growth). GH treatment can be given at a dosage of 0.35 mg/kg per week given six or seven times weekly.

Constitutional Delay of Growth

Constitutional delay of growth is characterized by normal prenatal growth followed by growth deceleration during infancy and childhood, and is reflected in declining height percentiles at this time. Between 3 years of age and late childhood, growth proceeds at a normal velocity. A period of pronounced growth deceleration can be observed immediately preceding the onset of puberty. Children with constitutional delay have later timing of puberty. At times, the combination of short stature accompanied and exaggerated by constitutional delay of growth and development in adolescents can cause sufficient psychosocial adolescent stress to warrant treatment with GH administered in the same manner and dosage as that used for treating GHD.

Cystic Fibrosis

Cystic Fibrosis (CF) is the most common lethal genetic disorder in America. An estimated 1000 individuals are born with Cystic Fibrosis each year in the United States. Cystic fibrosis causes dysfunction of the exocrine glands with increased viscosity of mucus secretions, which leads to pulmonary disease, exocrine pancreatic insufficiency, and intestinal obstruction. Early diagnosis and treatment has significantly decreased mortality in children with CF. However, malnutrition and poor growth continue to be a significant problem. Poor weight gain, weight loss, and inadequate nutrition result from reduced energy intake, increased energy loss, and increased energy expenditure. It has been reported that 28% of persons with CF are below the 10th percentile for height and 34% are below the 10th percentile for weight. Studies have shown that GH therapy improves height velocity, weight velocity, lean body mass (LBM) and pulmonary function in patients with cystic fibrosis.

Skeletal Dysplasias

Skeletal dysplasias associated with short stature such as achondroplasia can be treated with GH. Achondroplasia is a genetic disorder, affecting the fibroblast growth factor receptor type III gene, which is evident at birth. It affects about one in every 20,000 births and it occurs in all races and in both sexes. During fetal development and childhood, cartilage normally develops into bone, except in a few places, such as the nose and the ears. In individuals with achondroplasia the rate at which cartilage cells in the growth plates of the long bones turn into bone is slow, leading to short bones and reduced height.

Achondroplasia is characterized by short stature, short limbs, proximal extremity (upper arm and thigh), head appears disproportionately large for body, skeletal (limb) abnormalities, abnormal hand appearance (trident hand) with persistent space between the long and ring fingers, marked kyphosis and lordosis (spine curvatures), waddling gait, bowed legs, prominent (conspicuous) forehead (frontal bossing), hypotonia and polyhydramnios (present when affected infant is born). GH has been approved to treat achondroplasia in some countries such as Japan and South Africa but does not yet have FDA approval.

Intrauterine Growth Retardation (IUGR) and Children of Small Gestational Age (SGA Children)

GH treatment can be beneficial in children with inter uterine growth retardation or infants who are small for gestational age (a condition also termed Russell-Silver syndrome). One definition of inter uterine growth retardation is a weight below the 10^{th} percentile for gestational age or a birth weight 2 standard deviations below the mean for gestational age. Studies have shown that those children who don't show catch-up growth can benefit from GH treatment.

The present invention resides in the surprising finding that co-administration of GH with FFA regulators ameliorates the deterioration of insulin sensitivity through prevention of lipolysis, has decreased oedemic effects in comparison with the GH therapy alone and exerts synergism to increase linear growth above that of GH alone.

The invention provides a new method and a composition aimed at alleviating the conditions associated with GH therapy and enhancing the efficacy of the methods existing in the prior art. Moreover, the novel application disclosed in the invention provides the public with a beneficial alternative to the methods existing in the prior art.

Methods of Treatment

The invention in broad terms is directed to the treatment or prophylaxis of consequences of growth hormone (GH) treatment. GH is commonly used to treat conditions resulting in short stature including but not restricted to growth hormone insufficiency, growth hormone deficiency, Intrauterine Growth Retardation (Silver-Russell Syndrome), skeletal abnormalities, chromosomal variations (Turner's syndrome, Down syndrome), or chronic kidney disease related growth retardation. GH treatment has been shown to contribute to a number of conditions, as described earlier. Such conditions have also been observed to extend beyond immediate GH treatment. The applicants established that such consequences can at least be mitigated, if not completely prevented, by administration of a FFA regulator, preferably in combination with the GH treatment. The addition of FFAs to GH corrects insulin sensitivity to either the pre-treatment state or to that of normal children. Where the adverse consequences of growth hormone treatment have not been observed as symptoms, the incidence of the consequences can at least be mitigated prophylactically.

Of particular advantage is that while the adverse effects of the GH therapy are alleviated, the growth increase effect of the GH is enhanced by the use of the FFA regulator.

As a result, the combination treatment provides a useful method of treating the short stature condition (with administration of GH) while at the same time at least reducing some of the adverse consequences of the treatment.

Pharmaceutical Composition

In general, compounds of this invention will be administered as pharmaceutical compositions by one of the following routes: oral, topical, systemic (e.g. transdermal, intranasal, intrapulmonary or by suppository), parenteral (e.g. intramuscular, subcutaneous, intra-arterial, intraperitoneal or intravenous injection), by implantation and by infusion through such devices as osmotic pumps, transdermal patches and the like. Compositions may take the form of tablets, pills, capsules, cachets, lozenges, granules, semisolids, powders, sustained release formulation, solutions, suspensions, emulsions, elixirs, aerosols or any other appropriate compositions; and may include pharmaceutically acceptable excipients. Suitable excipients are well known to persons of ordinary skill in the art, and they, and the methods of formulating the compositions, may be found in such standard references as Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; Kibbe, et al., Eds., Handbook of Pharmaceutical Excipients (3rd Ed.), American Pharmaceutical Association, Washington, 1999; and Gennaro A R: Remington: The Science and Practice of Pharmacy, 20^{th} Ed., Lippincott, Williams and Wilkins, Philadephia, Pa. (2000). Suitable liquid carriers, especially for injectable solutions include water, aqueous saline solution, aqueous dextrose solution and the like, with isotonic solutions being preferred for intravenous administration.

The active compounds (GH and FFA regulator(s)) to be used in the treatment or prophylaxis in methods of the invention will be formulated and dosed in a fashion consistent with good medical practice, taking into account the clinical condition of the individual subject (especially the side effects of treatment with GH alone), the site of delivery of the composition(s), the method of administration, the scheduling of administration, and other factors known to practitioners. It is understood, that the specific dose level of each active compound (GH and FA regulator(s)) for each patient will depend upon a variety of factors including the activity of the specific agents employed, the age, body weight, general health, sex, diet, time of administration, rate of excretion, active agent combination selected, the severity of the particular conditions or disorder being treated, and the form of administration. The “effective amounts” of each component for purposes herein are thus determined by such considerations and are amounts that achieve the desired effects, said desired effects include but are not limited to increasing the growth rates of the subjects and/or reducing and/or preventing adverse consequences of GH treatment, especially deteriation of insulin sensitivity, oedema and/or trabecular bone loss. Appropriate dosages can be determined in trials.

Administration of FFA Regulators

In general, the daily dose of fibrates is usually in the range of 0.1 mg-100 mg/kg, typically 0.1-20 mg/kg. An intravenous dose may, for example, be in the range of 0.01 mg to 0.1 g/kg, typically 0.01 mg to 10 mg/kg, which may conveniently be administered as an infusion of from 0.1 μg to 1 mg, per minute. Infusion fluids suitable for this purpose may contain, for example, from 0.01 μg to 0.1 mg, per millilitre. Unit doses may contain, for example, from 0.1 μg to 1 g of each component. Thus ampoules for injection may contain, for example, from 0.1 μg to 0.1 g and orally administrable unit dose formulations, such as tablets or capsules, may contain, for example, from 0.1 mg to 1 g. Preferably, fibrates, particularly fenofibrate, are administered in an amount from about 50 to 450 mg daily.

A total daily dose of nicotinic acid or a nicotinic acid derivative can generally be in the range of from about 500 to about 10,000 mg/day in single or divided doses, or about 1000 to about 8000 mg/day, or about 3000 to about 6000 mg/day in single or divided doses.

Preferably, the nicotinic acid or a nicotinic acid derivative is administered orally. Orally administrable unit dose formulations, such as tablets or capsules, can contain, for example, from about 50 to about 500 mg, or about 200 mg to about 1000 mg, or from about 500 to about 3000 mg, of the nicotinic acid or nicotinic acid derivative.

Oral delivery of the nicotinic acid or nicotinic acid derivatives of the present invention can include formulations, as are well known in the art, to provide immediate delivery or prolonged or sustained delivery of the drug to the gastrointestinal tract by any number of mechanisms. Immediate delivery formulations include, but are not limited to, oral solutions, oral suspensions, fast-dissolving tablets or capsules, disintegrating tablets and the like. Prolonged or sustained delivery formulations include, but are not limited to, pH sensitive release from the dosage form based on the changing pH of the gastrointestinal tract, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester. Non-limiting examples of formulations, including extended release formulations, as found in NIASPAN® tablets (Kos Pharmaceuticals), are disclosed in U.S. Pat. No. 6,080,428 and U.S. Pat. No. 6,129,930, both incorporated herein by reference.

Administration of GH

Preferably, the effective amount of GH administered to a subject is between about 0.001 mg/kg/day and about 0.2 mg/kg/day; more preferably, the effective amount of GH is between about 0.01 mg/kg/day and about 0.1 mg/kg/day. In other aspects, the effective amount of GH administered to a subject is at least about 0.2 mg/kg/week. In another aspect, the effective amount of GH is at least about 0.25 mg/kg/week. In another aspect, the effective amount of GH is at least about 0.3 mg/kg/week. In one embodiment, the dose of GH ranges from about 0.3 to 1.0 mg/kg/week, and in another embodiment, 0.35 to 1.0 mg/kg/week. Preferably, the growth hormone is formulated at a pH of about 7.4 to 7.8.

Preferably, the GH is administered once per day subcutaneously. In preferred aspects, the dose of GH is between about 0.001 and 0.2 mg/kg/day. Yet more preferably, the dose of GH is between about 0.010 and 0.10 mg/kg/day.

The GH is suitably administered continuously or non-continuously, such as at particular times (e.g., once daily) in the form of an injection of a particular dose, where there will be a rise in plasma GH concentration at the time of the injection, and then a drop in plasma GH concentration until the time of the next injection. Another non-continuous administration method results from the use of PLGA microspheres and many implant devices available that provide a discontinuous release of active ingredient, such as an initial burst, and then a lag before release of the active ingredient. See, e.g., U.S. Pat. No. 4,767,628.

The GH may also be administered so as to have a continual presence in the blood that is maintained for the duration of the administration of the GH. This is most preferably accomplished by means of continuous infusion via, e.g., mini-pump such as an osmotic mini-pump. Alternatively, it is properly accomplished by use of frequent injections of GH (i.e., more than once daily, for example, twice or three times daily).

In yet another embodiment, GH may be administered using long-acting GH formulations that either delay the clearance of GH from the blood or cause a slow release of GH from, e.g., an injection site. The long-acting formulation that prolongs GH plasma clearance may be in the form of GH complexed, or covalently conjugated (by reversible or irreversible bonding) to a macromolecule such as one or more of its binding proteins (WO 92/08985) or a water-soluble polymer selected from PEG and polypropylene glycol homopolymers and polyoxyethylene polyols, i.e., those that are soluble in water at room temperature. Alternatively, the GH may be complexed or bound to a polymer to increase its circulatory half-life. Examples of polyethylene polyols and polyoxyethylene polyols useful for this purpose include polyoxyethylene glycerol, polyethylene glycol, polyoxyethylene sorbitol, polyoxyethylene glucose, or the like. The glycerol backbone of polyoxyethylene glycerol is the same backbone occurring in, for example, animals and humans in mono-, di-, and triglycerides.

The polymer need not have any particular molecular weight, but it is preferred that the molecular weight be between about 3500 and 100,000, more preferably between 5000 and 40,000. Preferably the PEG homopolymer is unsubstituted, but it may also be substituted at one end with an alkyl group. Preferably, the alkyl group is a C1-C4 alkyl group, and most preferably a methyl group. Most preferably, the polymer is an unsubstituted homopolymer of PEG, a monomethyl-substituted homopolymer of PEG (mPEG), or polyoxyethylene glycerol (POG) and has a molecular weight of about 5000 to 40,000.

Specific methods of producing GH conjugated to PEG include the methods described in U.S. Pat. No. 4,179,337 on PEG-GH and U.S. Pat. No. 4,935,465, which discloses PEG reversibly but covalently linked to GH, and also PEG-hGH conjugates as disclosed in WO99/03887, WO03/044056 and in WO2004/22630.

The GH can also be suitably administered by sustained-release systems. Examples of sustained-release compositions useful herein include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman et al., Biopolymers, 22, 547-556 (1983), poly(2-hydroxyethyl methacrylate) (Langer et al., J. Biomed. Mater. Res., 15: 167-277 (1981); Langer, Chem. Tech., 12: 98-105 (1982), ethylene vinyl acetate (Langer et al., supra) or poly-D-(−)-3-hydroxybutyric acid (EP 133,988), or PLGA microspheres.

Sustained-release GH compositions also include liposomally entrapped GH. Liposomes containing GH are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese Pat. Appln. 83-118008; U.S. Pat. Nos. 4,485,045 and 4,544,545; and EP 102,324. ordinarily, the liposomes are of the small (about 200-800 Angstroms) unilamellar type in which the lipid content is greater than about 30 mol. percent cholesterol, the selected proportion being adjusted for the optimal therapy. In addition, a biologically active sustained-release formulation can be made from an adduct of the GH covalently bonded to an activated polysaccharide as described in U.S. Pat. No. 4,857,505. In addition, U.S. Pat. No. 4,837,381 describes a microsphere composition of fat or wax or a mixture thereof and GH for slow release.

For parenteral administration, in one embodiment, GH is formulated generally by mixing the GH at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides. Generally, the formulations are prepared by contacting the GH with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or non-ionic surfactants such as polysorbates, poloxamers, or PEG.

GH is typically formulated individually in such vehicles at a concentration of about 0.1 mg/mL to 100 mg/mL, preferably 1-10 mg/mL, at a pH of about 4.5 to 8. GH is preferably at a pH of 7.4-7.8. It will be understood that use of certain of the foregoing excipients, carriers, or stabilizers will result in the formation of GH salts.

The foregoing describes the invention including preferred forms thereof. Alterations and medications that would be apparent to the skilled person are intended to be included within the spirit and scope of the invention disclosed.

Pharmacological Study 1

A study to assess the effectiveness of a combination therapy comprising GH and FFA regulators in improving linear growth and reducing metabolic abnormalities associated with GH therapy.

Study Design

This study utilised a well-characterised rodent model of short stature due to fetal growth retardation (Woodall et al. 1996).

A schematic of the overall experimental design is presented below.

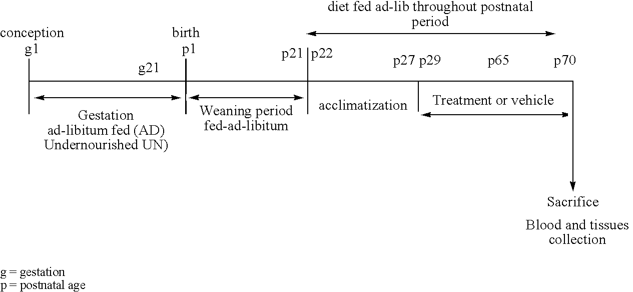

Test Groups—10 animals per group

| | |
|---|---|
| SGA (UN) | Ad Libitum (AD) |
| Control | Control |
| GH (5 mg/kg/day) | GH (5 mg/kg/day |
| GH (5 mg/kg/day) + fenofibrate | GH (5 mg/kg/day + fenofibrate |
| (30 mg/kg/day) | (30 mg/kg/day) |
| GH (5 mg/kg/day + acipimox | GH (5 mg/kg/day + acipimox |
| (20 mg/kg/day) | (20 mg/kg/day) |

Experimental Procedure—Methods and Analytical Procedures

Animal Model

The rodent model of maternal undernutrition used to induce SGA was initially characterised in the Liggins Institute, Faculty of Medical and Health Sciences, University of Auckland by Woodall et al. (1996). This model has since been published in several international peer-reviewed journals (Woodall et al. 1996, 1998; Vickers et al. 2000, 2001).

This experimental approach to induce SGA results in 30-35% growth retardation in 22-day old fetuses and persistent postnatal growth failure and no evidence of catch-up growth until at least 90 days of age. These animals develop hypertension, insulin resistance and truncal obesity as adults.

Animal Protocol to Generate SGA Offspring

Virgin Wistar rats (age 75-100 days) were time mated using a rat estrous cycle monitor (Fine Science Tools INC., North Vancouver, BC, Canada) to assess the stage of estrous of the animals prior to introducing the male. Day 1 of pregnancy was determined by the presence of spermatozoa in a vaginal smear. After confirmation of mating, rats were housed individually in standard rat cages containing wood shavings as bedding with free access to water. The animal room was maintained at 25° C. with a 12 hour light: 12 hour dark cycle. Dams were randomly assigned to receive food either ad-libitum (AD group and dams for cross fostering) or to receive 30% of ad-libitum (UN-group, determined by measuring food intake on the previous day of an ad-libitum fed dam). The diet composition was protein 18%, fat 4%, fibre 3%, ash 7% and carbohydrate 58% (Diet 86, Skellerup Stock Foods, Auckland, New Zealand). Food intake and body weight was recorded daily. Following birth, UN offspring were cross fostered onto ad-libitum fed mothers. Cross fostering is necessary due to lactational insufficiency in restricted fed dams. Litter size was adjusted to 8 pups per litter to assure adequate and standardised nutrition. Body weight of all pups was recorded daily. At weaning (age 21 days) pups were sexed, weight-matched and housed in pairs in standard cages. All animals were fed ad-libitum for the remainder of the study. Dams were sacrificed by CO₂ asphyxiation and excess pups by decapitation. All animal ethics were approved by the Animal Ethics Committee at the University of Auckland.

In this experiment, male offspring only were used.

The use of power calculations determined that group sizes of 10 were necessary to demonstrate statistically significant differences anticipated in body length and fasting insulin concentration.

Test Compounds

Recombinant Bovine Growth Hormone (rbGH)

Many studies in rodents utilize treatment with human GH (hGH) due to its relative ease of availability for experimental use. However, hGH possesses both lactogenic and somatogenic properties in the rat due to hGH binding to both prolactin receptors and GH receptors. This has been clearly documented in binding studies using hGH, bGH, oPRL and rat growth hormone (rGH) and rat prolactin (rPRL). Rat hepatocytes contain two types of binding sites that bind hGH. The first, somatogenic binding sites, are specific for the growth-promoting hormones bGH and rGH. The second, lactogenic, are specific for lactogenic hormones, oPRL and rPRL. Human GH has been shown to bind to both sites (Ranke et al., 1976).

Recombinant rat GH was not available in sufficient quantities for large-scale animal experiments. Therefore bGH, a pure somatogen in the rat and an agent which is not a ligand for the rat prolactin receptor (Yamada et al, 1984), was used in the study.

Animals were treated with bGH by subcutaneous injection at a dose of 5 mg/kg/kday and a volume of 100 ul. This was administered as a split dose (2×2.5 mg/kg/day) at 0800 and 1700h using a fine gauge diabetic syringe. Control animals were administered saline using an identical treatment protocol.

Fibrates

Fenofibrate belongs to the class of fibrates (fibric acid derivative drugs). Fibrates are hypolipidemic agents that efficiently lower serum triglyceride levels through mediation of the peroxisome proliferator-activated receptor-α (PPAR-α). In addition, fibrates are known to lower serum cholesterol levels.

Fenofibrate was administered by daily oral gavage (0800h) at a dosage of 30 mg/kg body weight/day.

Acipimox

Acipimox is a potent long-acting nicotinic acid (NA) analog. As a hypolipidaemic agent acipimox reduces serum concentrations of triglycerides and non-esterified fatty acids. Acipimox has been shown to partially prevent GH induced insulin resistance by inhibition of lipolysis (Segerlantz et al. 2001). Acipimox (Pharmacia) was administered by daily oral gavage (0800h) at a dose of 20 mg/kg body weight/day (Blachere et al. 2001).

Observations

Body Weight

Animals were weighed between 8-9 am every day for the duration of the experiment. Individual animals were observed daily for any signs of clinical change, reaction to treatment or ill health. There were no indications whatsoever of any adverse stress response and related symptoms in any of the treatment groups.

Food Consumption

Food intake was measured on a daily basis. Relative food intake per rat (grams consumed per gram body weight per day) was calculated using the amount of food given to and the amount of uneaten food left by each pair in each group.

Water Consumption

Water consumption was calculated daily by weighing water bottles at the same time on each day of the study.

Body Length

Body lengths (nose-anus and nose-tail) and bone length (tibial, femoral length) was assessed post-mortem using peripheral quantitative computed tomography (PQCT, Stratec) analysis. Bone density was also assessed via pQCT.

Blood Pressure

Systolic and diastolic blood pressure and heart rate were recorded by tail cuff plethysmography according to the manufacturer's instructions (Blood pressure analyser IITC, Life Science, Woodland Hills, Calif., USA). Rats were restrained in a clear plastic tube in a heated room (25-28° C.). After 10-15 minutes acclimatisation the cuff was placed on the tail and inflated to 240 mmHg. Pulses were recorded during deflation at a rate of 3 mmHg/sec and reappearance of a pulse was used to determine systolic blood pressure. A minimum of 3 clear systolic blood pressure recordings were taken per animal. Previous observations indicate that the coefficient of variation for repeated measurements is <5%.

Plasma Analyses

Blood samples were collected following overnight fast. Samples were collected from the tail vein and at termination following decapitation under halothane anaesthetic. Blood samples were collected into heparinised tubes and centrifuged for harvesting of plasma. Blood samples were then analysed for insulin, glucose, FFAs, leptin, IGF-I, glycerol, triglycerides, cholesterol, corticosterone, markers of hepatic function (ALT, AST, ALP), and for markers of protein synthesis.

Plasma FFAs, triglycerides and glycerol were measured by diagnostic kit (Boehringer-Mannheim #1383175 and Sigma #337 respectively). Plasma leptin, insulin were measured using commercially available kits (inco, St Charles, Mo., US). Plasma IGF-I was measured by RIA as described previously (Vickers et al., 2000). Plasma glucose concentrations were measured using a colorimetric plate assay. All other plasma analytes (liver enzymes, electrolytes, etc.) were measured by a BM/Hitachi 737 analyser by Agriquality Laboratory Services (Auckland, New Zealand).

Tissue Studies

At termination animals were sacrificed by decapitation under halothane anaesthesia. Tissues (heart, liver, muscle and adipose (subcutaneous and visceral)) were collected, weighed and snap frozen in liquid nitrogen for subsequent analysis. An aliquot of liver tissue was also frozen at −20° C. for examining the growth hormone receptor using ligand-binding analysis.

Data Analysis

Data was analysed using multiple regression analysis or factorial ANOVA/ANCOVA with post hoc correction (prenatal influences and postnatal treatment effects) where appropriate. The statistical package utilised was StatView (Version 5, SAS Institute).

Previous data provided the basis of power calculations for the proposed studies (assuming α=0.05). For insulin sensitivity, an n of 10 will detect with a power of 80% a change of 0.2 and at 95% a change of 0.26 ng/ml with an SD of 0.15 ng/ml. For body length, an n of 10 will detect with a power of 80% a change of 6.88 mm and at 95% a change of 7.97 mm with an SD of 5.2 mm.

Results

There was a small reduction in maternal body weights compared to day 1 of gestation in pregnant SGA group females until day 15 of gestation. From day 15 of gestation, SGA dams gained weight and had achieved pre-mating weights by the time of parturition. Litter size was not significantly different between the two groups (AD 13.4±0.4, SGA 12.8±1.1). Maternal undernutrition resulted in fetal growth retardation reflected by significantly decreased body weight at parturition in the offspring from SGA dams (AD males 6.1±0.49 g, SGA 4.3±0.6 g, p<0.0001). Nose-anus (NA) and nose-tail (NT) lengths were significantly shorter at birth in SGA offspring compared to AD offspring (NA: AD males 49.3±2.43 mm, SGA males 44±3.0 mm; NT: AD males 65.9±2.8 mm, SGA males 58±4.1 mm, p<0.0001 for both lengths). From parturition until weaning at day 22, body weights remained significantly lower in the SGA offspring. At commencement of treatment, SGA offspring were significantly lighter than AD animals (p<0.0001) and total body weights remained significantly lower in SGA offspring for the remainder of the study.

Weight Response

Body weight gain (gain in grams) was significantly increased in all treatment groups (p<0.0001) compared to saline (FIG. 1). There was no significant difference in absolute body weight gain between animals treated with GH and the animals treated with either combination therapies. However, GH and acipimox treated animals had a significantly increased body weight gain compared to GH and fibrate treated animals. SGA animals were significantly lighter than AD animals for all treatment groups and there were no statistical interactions.

Compared to GH alone, AD animals treated with GH and acipimox showed a gradual divergence from GH alone animals in body weight gain (FIG. 1). However, the effect of the combination treatments in AD animals appeared to wane by about postnatal day 57 compared to GH treatment alone. In SGA animals, GH and fenofibrate combination therapy showed a marked increase in weight gain compared to GH treated animals but this effect waned after about 2 weeks of co-therapy and by the end of the trial these animals were growing at a slightly slower rate than GH treated animals. However, SGA animals treated with GH and acipimox showed a slow but positive weight gain increment compared to GH treated animals which had not abated at the end of the trial (FIG. 2).

Analysis of weight change per day also indicates that there is an acute beneficial effect of the combination therapies on body weight gain compared to GH alone. This is most marked in the GH and acipimox treated animals, in particular the SGA animals (FIG. 3).

Bone Length

Tibias were stored in 10% neutral buffered formalin. Tissue was stripped from the bone and bone length, area and density (cortical and trabecular) was assessed using pQCT (Stratec). Tibial length was significantly reduced in SGA offspring. GH significantly increased tibial length in all treated groups. However, GH and acipimox combination therapy enhanced the GH-induced effects on tibial growth (p<0.0001), FIG. 4). Tibial length in the GH and fenofibrate treated animals was not significantly different from that of GH alone. Tibial length was highly correlated with total body (nose-anus) length (FIG. 5). Total tibial area was significantly reduced in SGA animals and was increased in all treated animals.

Interestingly, GH treatment significantly reduced trabecular bone mass. However, this trabecular loss was not apparent in those AD and SGA animals treated with the combination therapy (Table 1).

TABLE 1
Fisher's PLSD for TRABECULAR
Effect: treatment
Significance Level: 5%

| | Mean Diff. | Crit. Diff | P-Value | |
|---|---|---|---|---|
| GH, GH/ACIP | −4.381 | 12.537 | .4868 | S |
| GH, GH/FIB | −10.613 | 12.537 | .0955 | |
| GH, SALINE | −14.137 | 12.537 | .0278 | |
| GH/ACIP, GH/FIB | −6.231 | 12.537 | .3237 | |
| GH/ACIP, SALINE | −9.756 | 12.537 | .1247 | |
| GH/FIB, SALINE | −3.525 | 12.537 | .5755 | |

The SSI (stress strain index) was significantly reduced in SGA animals and was increased in all GH/GH combination treated animals.

Total bone density was not significantly altered in any of the treatment groups although there was a trend (p=0.056) towards to drop in total bone density in the GH group which was not observed in the combination therapy groups. Cortical bone density (cortical and subcortical, mm²) was not significantly altered in any of the treatment groups.

Body Lengths

Nose anus and lengths were significantly increased with GH treatment and, moreover, were further increased using combination therapy with GH and acipimox (p<0005 for GH versus GH and acipimox) (FIG. 6).

Body Mass Index (BMI)

A BMI was calculated using: body weight/nose-anus length (cm)₂. BMI was significantly lower in SGA animals compared to AD animals (p<0.05). BMI was significantly reduced in GH and acipimox treated animals compared to both saline and GH treated animals (p<0.005). BMI was not significantly different between saline and GH treated animals. Due to the lack of lipolysis in the GH and acipimox treated animals compared to GH treated, alterations in BMI probably reflect an enhancement of liner growth above that of GH alone.

Food Intake

There was no significant difference in relative food intake (grams consumed per g body weight) in any of the treatment groups. SGA animals were hyperphagic with a slight but significantly increased food intake compared to AD animals (p<0.05) which concurs with our previous observations.²

Water Intake

There were no significant differences in water intakes between any of the treatment groups. However, there was a trend (p=0.09) towards an increase in relative water intake (water consumed per g body weight) in the GH plus acipimox treated groups, particularly in the AD animals. SGA animals had a slightly but significantly (p<0.05) lower relative water intake compared to AD animals.

Blood Hematocrit

A well-characterised effect of GH treatment is increased plasma volume (Johannsson et al, 2002). Decrease in blood hematocrit is a reliable marker of increase in plasma volume associated with fluid retentive effects of GH therapy. As expected, blood plasma hematocrit was significantly reduced in GH treated animals in both AD and SGA groups. The decrease in hematocrit was also observed in the GH and fenofibrate treated animals, but, surprisingly, there was no effect of the GH and acipimox combination in lowering hematocrit. Plasma hematocrit was significantly higher in the GH and acipimox treated animals compared to the GH alone and GH and fenofibrate groups and was not significantly different from that of saline (FIG. 7), though the combination of GH and fibric acid derived FFA regulator displayed a degree of synergism in ameliorating GH-induced fluid retention.

Liver

Liver weight relative to body weight was not significantly different between AD and SGA animals. Relative liver weight was significantly increased in AD and SGA animals treated with GH and fenofibrate (FIG. 8). GH alone or in combination with acipimox had no effect on liver weight.

Retroperitoiteal Fat Depots

There was no significant difference between AD and SGA animals in relative retroperitoneal fat depots. Treatment with GH or GH and fenofibrate combination significantly reduced retroperitoneal fat mass compared to saline controls (FIG. 9). Retroperitoneal fat was significantly reduced with GH therapy but this lipolysis was partially blocked by combination therapy, particularly in SGA animals administered GH in combination with acipimox.

Kidneys

Kidney weights were significantly reduced relative to body weight in SGA animals compared to AD animals (p<0.005). Relative kidney weights were significantly increased in the GH+fenofibrate animals compared to all other treatment groups. Relative kidney weight was reduced in GH animals compared to saline controls but GH and acipimox treated animals were not significantly different from controls.

Adrenals

Adrenal weight was not significantly different between AD and SGA animals. Adrenal weights were significantly increased in all treatment groups compared to saline controls. Adrenal weight was significantly increased in the GH and fenofibrate as well as in GH and acipimox treated animals compared to those treated with GH alone (FIG. 10).

Spleen

Relative spleen weights were significantly increased in SGA animals compared to AD animals. Spleen weights were increased in all treatment groups relative to body weight and there was a trend towards further splenic growth in GH+fenofibrate animals compared to controls (p=0.056) (FIG. 11)

IGF-I

Plasma IGF-I was significantly increased in GH and in GH and acipimox combination treated AD and SGA animals compared to salien controls (FIG. 12). However, plasma IGF-I was not significantly elevated in the GH+fenofibrate treated animals. The rise in IGF-I in the GH treted animals animals was not significantly different from the IGF-1 ise seen and in the GH and acipimox treated animals.

Fasting Insulin

Fasting plasma insulin was significantly increased in the GH and fenofibrate treated animals compared to saline treated. Insulin concentrations were not significantly altered with the GH and acipimox treated animals but were significantly lower than those treated with GH alone or in combination with fenofibrate (FIG. 13). There was no significant difference in insulin levels between the AD and SGA animals.

Fasting Glucose

Fasting plasma glucose was not significantly different between AD and SGA animals and was not significantly altered by GH therapy (FIG. 14). Plasma glucose was significantly lower in the GH and acipimox treated animals compared to GH alone and there was an overall trend for glucose to be lower than controls in the GH and acipimox treated animals (p=0.07). Glucose in the GH and fenofibrate groups was significantly increased compared to saline and GH/GH and acipimox treated animals. There was no significant difference in glucose levels between the AD and SGA animals.

Leptin

There was no statistically significant difference in plasma leptin concentrations between AD and SGA animals (FIG. 15). Leptin was elevated in GH treated animals compared to saline animals and animals that received GH and fibrate. There was no difference in leptin concentrations between GH treated animals and those administered GH and acipimox.

Free Fatty Acids (FFAs)

Plasma FFAs were not significantly different between AD and SGA animals. Plasma FFAs were significantly reduced in AD and SGA animals treated with GH and acipimox compared to saline treated and animals treated with GH alone (FIG. 16). Interestingly, the GH and fibrate combination did not lower FFA concentrations and were significantly higher than those treated with GH and acipimox.

Triglycerides

Plasma triglycerides were not significantly different between AD and SGA animals (FIG. 17). Triglycerides were significantly lower in GH and acipimox treated animals compared to all other treatment groups. There was no significant effect of GH treatment on triglycerides compared to saline controls.

Free Glycerol

There was no difference in plasma glycerol between AD and SGA animals (FIG. 18). Plasma glycerol was significantly decreased in GH and acipimox treated animals compared to all other treatment groups. (FIG. 18)

Systolic Blood Pressure

As our group has shown previously, systolic blood pressure was significantly elevated in SGA animals (FIG. 19). Treatment of SGA offspring with GH or GH and FFA regulators significantly reduced and normalised systolic blood pressure (FIG. 20). This agrees with our previous reports on the anti-hypertensive effects of GH. (Vickers et al. 2002) Systolic blood pressure was normal in AD animals and there was no effect of treatment.

Discussion

The effects of combination therapy on body weight gain were as marked in normal animals, as they were in animals born of low birth weight. However, with regard to the GH and acipimox combination therapy in AD animals, weight gain plateaued during the trial compared to GH treated animals. This waning of dose efficacy was not observed in SGA animals where there was a clear divergence in body weight gain compared to GH treated animals as the trial progressed.

We have unexpectedly found that the synergistic combination therapy consisting of GH and nicotinic acid derived FFA regulator, acipimox, significantly enhanced linear growth above that of GH alone or GH in combination with fenofibrate.

GH monotherapy and GH combination therapy increased bone length in all treatment coups in comparison with controls. We have found that GH and acipimox combination treatment markedly enhanced the GH effects on tibial growth and achieved greater increase in tibial length than GH in combination with fenofibrate.

Additionally we have discovered that both combination treatments reduced trabecular bone loss associated with GH monotherapy.

We have unexpectedly found that the combination therapy consisting of GH and nicotinic acid derived FFA regulator, acipimox, had a beneficial effect on the plasma volumes in the treatment group, in comparison with animals treated with GH or GH in combination with fibric acid derived FFA regulator. In the GH and acipimox treated group there was no increased in plasma volume associated with GH monotherapy.

SGA animals had elevated blood pressure compared to AD animals. Systolic blood pressure was normalized in this group using either GH alone or a combination approach which agrees with our previous patented observations.

In summary, GH and acipimox therapy enhanced linear growth above that of GH alone and ameliorated the fluid retentive effects normally associated with GH therapy. The combination of GH and fenofibrate was less effectual than that of GH and acipimox. We observed metabolic benefits of GH and acipimox co-therapy (including improved insulin sensitivity and blockage of lipolytic effects induced by GH treatment i.e. pharmacological anti-lipolysis) over GH monotherapy.

### BIBLIOGRAPHY

Azcona C, Albanese A, Bareille P, Stanhope R. 1998. Growth hormone treatment in growth hormone-sufficient and -insufficient children with intrauterine growth retardation/Russell Silver Syndrome. Hormone Research 50: 22-7.
Barker D. Mothers, babies and diseases in later life. BMT Publishing Group, 1994.
Barker D J, Hales C N, Fall C H, Osmond C, Phipps K, Clark P M. 1993 Type 2 (non-insulin-dependent) diabetes mellitus, hypertension and hyperlipidemia (Syndrome X): relation to reduced foetal growth. Diabetologia 36: 62-7.
Breier, B. H., Gluckman, P. D., and Bass, J. J. The somatotrophic axis in young steers: Influence of nutritional status and oestradiol 17-B on hepatic high and low affinity somatotrophic binding sites. Journal of Endocrinology 1988; 116, 169-177.
Caprio S, Boulware S, Diamond M, Sherwin R S, Carpenter T O, Rubin K, Amiel S, Press M, Tamborlane W V. 1991 isulin Resistance: an early metabolic defect of Turner's syndrome. J. Clin. Endocrinol. Metab. 1991 72 832-6.
Cross D A, Alessi D R, Cohen P, Andjelkovich M, Hemmings B A. inhibition of glycogen synthase kinase-3 by insulin mediated by protein kinase B. Nature 1995; 378:785-89.
Cutfield W S, Hofman P L, Jackson W E, Rolfe G, Robinson E M, Breier B H, Vickers M. Reduced insulin sensitivity during GH therapy in IUGR children. Oral presentation at International Congress of Endocrinology 2000. Sydney, Australia November 2000 (2)
Cutfield W S, Wilton P, Bennmarker H, Albertsson-Wikland K, Chatelain P, Ranke M B, Price D A. 2000. The incidence of diabetes mellitus and impaired glucose tolerance in children and adolescents receiving growth hormone treatment. The Lancet; 355: 610-13. (1)
DeZegher F, Albertsson-Wikland K, Wollman H A, Chatelain P, Chaussain J L, Lofstrom A et al. 2000. Growth hormone treatment of short children born small for gestational age: growth responses with continuous and discontinuous regimens over six years. Journal of Clinical Endocrinology and Metabolism 85: 2816-21.
DeZegher F, Maes M, Gargosky S E, Heinrichs C, Du Caju M U L, Thiry G et al. 1996. High dose growth hormone treatment of short children born small for gestational age. Journal of Clinical Endocrinology and Metabolism 81: 1887-92.96
Dudley D T, Pang L, Decker S J, Bridges A J, Saltiel A R. A synthetic inhibitor of the mitogen-activated protein kinase cascade. Proc Natl Acad Sci 1995; 92:7686-89.
Felber J P, Vannotti A. 1964 Effect of fat infusion on glucose tolerance and insulin plasma levels. Medical Experimentation 10: 153-7.
Feldman R D, Briebier G S. 1993 Insulin-mediated vasodilation: impairment with increased blood pressure and body mass. Lancet 342 707-9.
Fjelstad-Paulsen A, Czernichow P, Bost M, Colle M, Lebouc J Y, Lecornu M, Leheup B, Lima J M, Raux M C, Toublanc J E, Rappaport R. 1998. Three year data from a comparative study with recombinant growth hormone in the treatnent of short stature in young children with intrauterine growth retardation. Acta Paediatrica 87: 511-7.
Guerre-Millo M et al. 2000. Peroxisome Proliferator-activated Receptor CZ Activators Improve Insulin Sensitivity and Reduce Adiposity. J. Biol. Chem. 275(22); 16638-16642.
Hofman P L, Cutfield W S, Robinson E M, Bergman R N, Menon R K, Sperling M A, Gluckman P D. 1997 Insulin resistance in short children with intrauterine growth retardation. Journal of Clinical Endocrinology and Metabolism 82: 402-6.
Kahn S E, Prigeon R L, McCulloch D K Boyko E J, Bergman R N, Schwartz M W, Neifing J L, Ward W K, Beard J C, Palmer J C. 1993 Quantification of the relationship between insulin sensitivity and beta cell function in human subjects. Diabetes 42: 1663-72.
Laakso M, Edelman S V, Brechtel G, Baron A D. 1990 Decreased effect of insulin to stimulate skeletal muscle blood flow in obese man. Journal of Clinical Investigation 85: 1844-52.
Laakso M, Edelman S V, Brechtel G, Baron A D. 1992 Impaired insulin-mediated skeletal muscle blood flow in patients with NIDDM. Diabetes 41: 1076-83.
Law C M, Barker D J, Bull A R, Osmond C. 1991 Maternal and foetal influences on blood pressure. Archives of Diseases in Childhood 66: 1291-95.
Lowry, O. H., Rosebrough, N. J., Farr, A. L., and Randall, R. J. Protein measurement with the folin phenol reagent. Journal of Biological Chemistry 1951; 193, 265-275. 1951.
Martin B C, Warram J H, Krolewski A S, Bergman R N, Soeldner J S, Kahn C R. 1992 Role of glucose and insulin resistance in development of type 2 diabetes mellitus: results of a 25-year follow-up study. Lancet 1992 340: 925-9.
Moller N, Jorgensen A O L, Abildgard L et al. 1991 Effects of GH on glucose metabolism. Hormone Research; 36 (Suppl 1): 32-5.
Nielsen S, Møller N, Pedersen S B, Christiansen J S, Jorgensen J O L. The effect of long-term pharmacological antilipolysis on substrate metabolism in growth hormone (GH)-substituted GH-deficient adults. J Clin Endocrinol Metab 2002; 87:3274-78.
Ozanne S E, Dorling M W, Wang C L, Nave B T. Impaired PI 3-kinase activation in adipocytes from early growth-restricted male rats. Am J Physiol Endocrinol Metab 2001; 280:E534-E539.
Ozanne S E, Dorling M W, Wang C L, Nave B T. Impaired PI 3-kinase activation in adipocytes from early growth-restricted male rats. Am J Physiol Endocrinol Metab 2001; 280:E534-E539.
Ozanne S E, Nave B T, Wang C L, Shepherd P R, Prins J, Smith G D. Poor fetal nutrition causes long-term changes in expression of insulin signalling components in adipocytes. Am J Physiol 1997; 273: E46-E51. Ranke, M. B., Stanley, C. A., Tenore, A., Rodbard, D., Bongiovanni, A. M. and Parks, J. S. Endocrinology (Baltimore) 1976; 99, 1033-1045
Randle P J, Garland P B, Hales C N, Newsholme E A. 1963 The glucose fatty acid cycle. Its role in insulin sensitivity and the metabolic disturbances of diabetes mellitus. Lancet 1: 785-789.
Ranke M B, Lindberg A. Acta Paediatr 1996; 85 [Suppl 417]: 18-26.
Ranke, M. B., Stanley, C. A., Tenore, A., Rodbard, D., Bongiovanni, A. M. and Parks, J. S. Endocrinology (Baltimore) 1976; 99, 1033-1045.
Reaven G, Chang H, Hoffman B B. 1988 Additive hypoglycemic effects of drugs that modify free-fatty acid metabolism by different mechanisms in rats with streptozocin-induced diabetes. Diabetes 37: 28-32.
Reaven G M. 1991. Resistance to insulin-stimulated glucose uptake and hyperinsulinemia: role in non-insulin-dependent diabetes, high blood pressure, dyslipidemia and coronary heart disease. Diabetes and Metabolism. 17(1 Pt 2):78-86.
Rosenfeld R G, Attie K M, Frane J et al. J Pediatr 1998; 132: 319-24.
Segerlantz M., Bramnert M., Manhem P., Laurila E., Groop L. C. Inhibition of the rise in FFA by Acipimox partially prevents GH-induced insulin resistance in GH-deficient adults. J Clin Endocrinol Metab 2001; 86(12):5813-8.
Singh, K., Ambler, G. R., Breier, B. H., Klempt, M., and Gluckman, P. D. Ovine placental lactogen is a potent somatogen in the growth hormone (GH)-deficient rat: comparison of somatogenic activity with bovine GH. Endocrinology 1992, 130, 2758-2766
Sugimoto M, Takeda N, Nakashima K, Okumura S, Takami K, Yoshino K, Hattori J, Ishimori M, Takami R, Sasaki A, Yasuda K. 1998 Effect of troglitazone on hepatic and peripheral insulin resistance induced by growth hormone excess in rats. Metabolism 47: 783-7.
Thorell, J. I. and Johansson, B. G. Enzymatic iodination of polypeptide hormones with 125I to high specific activity. Biochimica et Biophysica Acta 251, 363-369.1971.
Vickers M H, Breier B H, Cutfield W S, Hofman P L, Gluckman P D. Fetal origins of hyperphagia, obesity and hypertension and its postnatal amplification by hypercaloric nutrition. Am J Physiol 2000; 279:E83-E87.
Vickers M H, Ikenasio B A, Breier B H. IGF-1 treatment reduces hyperphagia, obesity, and hypertension in metabolic disorders induced by fetal programming. Endocrinology 2001; 142:3964-73.
Vickers M H, Reddy S, Ikenasio B A, Breier B H. Dysregulation of the adipoinsular axis—a mechanism for the pathogenesis of hyperleptinemia and adipogenic diabetes induced by fetal programming. J Endocrinol 2001; 170:323-32.
Vickers M H, Ikenasio B A, Breier B H. Adult growth hormone treatment reduces hypertension and obesity induced by an adverse prenatal environment. J Endocrinol 2002; 175:615-23.
Walker K S, Deak M, Paterson A, Hudson K, Cohen P, Alessi D R. Activation of protein kinase B beta and gamma isoforms by insulin in vivo and by 3-phosphoinositide-dependent protein kinase-1 in vitro: comparison with protein linase B alpha. Biochem J 1998; 331:299-308.
Woodall S M, Bassett N S, Gluckman P D, Breier B H. Consequences of maternal undernutrition for fetal and postnatal hepatic insulin-like growth factor-I, growth hormone receptor and growth hormone binding protein gene regulation in the rat. Journal of Molecular Endocrinology 1998; 20:313-26.
Woodall S M, Breier B H, Johnston B M, Gluckinan P D. A model of intrauterine growth retardation caused by chronic maternal undernutrition in the rat: effects on the somatotropic axis and postnatal growth. J Endocrinol 1996; 150:231-42.
Woodall S M, Johnston B M, Breier B H, Gluckman P D. Chronic maternal undernutrition in the rat leads to delayed postnatal growth and elevated blood pressure of offspring. Pediatr Res 1996; 40:438-43.
Yamada, K. and Donner, D. B. Structures of the somatotropin receptor and prolactin receptor on rat hepatocytes characterized by affinity labelling. Biochem. J. 1984; 220, 361-369

1. A method of treating a growth disorder in a juvenile, said method comprising administering to said juvenile an effective amount of at least one FFA regulator in combination with growth hormone.

2. A method of increasing the growth promoting effects of growth hormone therapy in a juvenile, said method comprising administering an effective amount of at least one FFA regulator in combination with growth hormone.

3. A method of preventing or treating an adverse consequence of growth hormone treatment in a juvenile, comprising administering an effective amount of at least one FFA regulator in combination with said growth hormone treatment.

4. A method of preventing or treating oedema as an adverse consequence of growth hormone treatment in a mammal, comprising administering an effective amount of at least one FFA regulator in combination with said growth hormone treatment.

5. A method of preventing or treating trabecular bone loss associated with early stages of GH therapy as an adverse consequence of growth hormone treatment in a mammal, comprising administering an effective amount of at least one FFA regulator in combination with said growth hormone treatment.

6. (canceled)

7. (canceled)

8. The method of claim 1, wherein said growth disorder is selected from a group consisting of growth hormone insufficiency, growth hormone deficiency, Intrauterine Growth Retardation, prematurity, growth failure in children who were born small for gestational age, very low birth weight, skeletal abnormalities, chromosomal variations, chronic renal insufficiency related growth retardation, constitutional delay of growth, cystic fibrosis related growth retardation, idiopathic short stature, short stature due to glucocorticoid treatment in children, failure of growth catching for short premature children, or any other condition resulting in short stature.

9. The method of claims 1, 2, 3, 4, 5 or 8, wherein said FFA regulator is fibric acid, nicotinic acid, a fibric acid derivative or a nicotinic acid derivative.

10. The method of claims 9, wherein said FFA regulator is nicotinic acid or a nicotinic acid derivative.

11. The method of claim 10, wherein said FFA regulator is acipimox.

12. The method of claim 1, 2, 3, 4 or 5, wherein said GH is administered by subcutaneous injection.

13. The method of claim 1, 2, 3, 4 or 5 wherein said FFA regulator(s) is administered orally.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A composition for treating growth disorders and/or preventing or treating the adverse consequences of growth hormone treatment, comprising growth hormone and at least one FFA regulator.

27. The composition according to claim 26, wherein said composition further comprises a suitable pharmaceutical carrier and/or excipient for said growth hormone and/or said FFA regulator(s).

28. The composition of claim 26 or 27, wherein said FFA regulator is fibric acid or a fibric acid derivative.

29. The composition of claim 28, wherein said FFA regulator is fenofibrate.
